(19) United States
(12) Patent Application Publication
(10) Pub. No.: US 2020/0306400 A1
(43) Pub. Date: Oct. 1, 2020

(54) SILVER NANOPARTICLES IMPREGNATED COVERS FOR ELECTRONIC DEVICES TO COMBAT NOSOCOMIAL INFECTIONS

(71) Applicant: Infection Sciences, LLC, Mobile, AL (US)

(72) Inventors: Sumit Arora, Mobile, AL (US); Om Prakash Jha, Mobile, AL (US); Robert C. Ross, JR., Fairhope, AL (US); J. Mark Swanzy, Mobile, AL (US)

(73) Assignee: Infection Sciences, LLC, Mobile, AL (US)

(21) Appl. No.: 16/894,235

(22) Filed: Jun. 5, 2020

Related U.S. Application Data
(63) Continuation of application No. 15/902,566, filed on Feb. 22, 2018.
(60) Provisional application No. 62/600,486, filed on Feb. 22, 2017.

(51) Int. Cl. A61L 2/238; A01N 59/16

(52) U.S. Cl. CPC A61L 2/238; B82Y 30/00; A01N 59/16

(57) ABSTRACT

An anti-microbial covering for use with electronic devices used in a healthcare environment is disclosed. The covering is impregnated during manufacturing with nano-sized silver particles and wrapped around the electronic device to stop the spread of nosocomial infections. The silver particles vary in shape and size to maximize the anti-microbial effects of each sheet, and each particle is sized to be less than 60 nanometers in diameter. The covering may be used in a process to disrupt nosocomial infections in a healthcare facility by having employees use the covering to cover electronic devices in the facility. The process includes a method for tailoring the anti-microbial covering to target specific pathogens present in the facility or, alternatively, tailor the covering to target pandemic level threats, such as for example the Covid-19 virus or seasonal influenza viruses.

This application claims the benefit of filing priority under 35 U.S.C. § 119 and 37 C.F.R. § 1.78 of the co-pending U.S. Provisional Application Ser. No. 62/600,486 filed Feb. 22, 2017, for a Silver Nanoparticles Impregnated Covers for Electronic Communication Devices to Combat Nosocomial Infections, and co-pending U.S. nonprovisional application Ser. No. 15/902,566 filed Feb. 22, 2018, for Silver Nanoparticles Impregnated Covers for Electronic Devices to Combat Nosocomial Infections. All information disclosed in those prior pending applications are incorporated herein by reference.

### FIELD OF THE INVENTION

The present invention relates generally to materials providing anti-microbial properties. In particular, the invention relates to coverings for electronic devices integrated with an antimicrobial agent or compound. The invention further relates to materials impregnated with silver based substances to combat microbial activity.

### BACKGROUND OF THE INVENTION

Overall incidence of hospital-acquired infections (hereinafter referred to as “HAIs” or simply, “nosocomial infections”) in the USA is on the rise and contributes significantly to morbidity and mortality of patients. HAIs kill more people each year than breast cancer, prostate cancer and AIDS combined in USA making it the fifth leading cause of death in U.S. acute-care hospitals. These infections increase a patient's hospital stay on average from 4.5 to 21.1 days, and kill over 90,000 U.S. patients each year. Centers for Disease Control and prevention (CDC) has estimated that HAIs add an average of $57,000 to a patient's hospital bill and $28 billion to $45 billion added to the nation's healthcare costs each year. According to general medical literature, electronic communication devices (hereinafter “ECDs”) used in hospital are considered as major reservoirs for transmission of nosocomial infections. ECDs are used for communication in every location of the hospital including operating room and intensive care units. More than 70% of the bacteria that cause HAIs are drug resistant organisms. For example, Methicillin-Resistant Staphylococcus Aureus popularly known as “MSRA” is a well-known drug resistant bacterium present in most hospitals that costs lives each year.

Nanotechnology is the convergence of different sciences such as physics, chemistry, biology, material science and medicine, which finds large applications in multiple aspects of research and in everyday life. The availability of new nanomaterials has caused a rapid expansion of the medical arts, often referred to as “nanomedicine,” and are now incorporated into a range of products and technologies. These applications can be in general useful for the management of various microbial infections and in particular for diagnostic and therapeutic uses. Although we live in an era of advanced and innovative technologies for elucidating the underlying mechanism of diseases and creating molecular designs for new drugs, infectious diseases continue to be one of the greatest health challenges worldwide.

The widespread therapeutic use of the antimicrobial chemicals has resulted in bacterial resistance to antibiotics. However, metal nanoparticles have recently become known to be a promising antimicrobial agent that acts on a broad range of target sites on microorganisms, both extracellularly and intracellularly. Moreover, the advances in reducing ions to nanoscale-sized particles have enabled the integration of metal nanoparticles into a large number of materials such as plastics, coating materials, foams and fibers, both natural and synthetic. These nanomaterials have proven their effectiveness for treating infectious diseases, including antibiotic resistance, in vitro as well as in animal models.

Silver is well known for its antimicrobial properties. Silver derives its broad spectrum antimicrobial effect from its ability to bind irreversibly to a variety of nucleophilic groups commonly found in or on the cells of bacteria, viruses, yeast, fungi, and protozoa. Binding to cellular components disrupts the normal reproduction and growth cycle resulting in the death of a cell. Capitalizing on this potent activity, silver in its various compounds and formulations has historically been incorporated into a variety of wound care preparations, such as ointments, hydrogels, hydrocolloids, creams, gels, and lotions. Further, silver nanoparticles are currently being used on the surfaces of various consumer medical products, wound care supplies, and medical treatment supplies, including bandages, dressings, catheters, and sutures, and such usages have proven the safety of silver nanoparticles for human use. After adjusting for the range of effectiveness, the benefits of such infection prevention is expected to be valued at more than $32 billion during the next decade.

According to the medial literature, electronic communication devices or electronic medical devices (hereinafter “ECDs” or “EMDs”) used in hospitals can become major reservoirs for transmission of harmful microorganisms and nosocomial infections. Today, ECDs such as for example, mobile phones, pagers, conference phones, and electronic tablets such as iPads®, have become indispensable accessories of professional and social life among doctors and other health care workers in hospitals. In fact, electronic tablets have quickly become an indispensable device for patient record reviews and updating of patient records during patient exams and procedures. EMDs and ECDs are used for all types of activities and are present in every location of a hospital, including operating rooms and intensive care units. In contrast to the expected benefits of the these devices, EMDs and ECDs are seldom cleaned, but are frequently touched during or after the examination of patients without hand-washing, and have been proven to act as reservoirs for transmission of nosocomial infections. Colonization of potentially pathogenic organisms on EMDs and ECDs has been reported in the literature. Once colonized on the surface of these EMDs and ECDs, infectious microbes can survive for extended periods, unless, these are eliminated by disinfection or sterilization procedure. The United States of America has one of the largest telecommunication networks in the world, and the medical community as with the rest of U.S. society is fully dependent on its telecommunication networks for efficiency. However, despite this efficiency and the known burden of HAIs in hospitals across the U.S., and the growing threat of antibiotic resistant pathogens, no disinfection guidelines have been adopted or issued by the CDC for EMDs or ECDs to reduce nosocomial infections.

While demand for nanoparticles-enhanced products has increased over time, developing techniques for integrating silver nanoparticles into the substrate of products, and in particular EMD or ECD products has remained a challenge. Current approaches result in inefficient use of high value materials, and although there are many approaches to attach nanoparticles to various substrates, those techniques have generally failed to ensure the effectiveness of such nanoparticles or that they remain affixed to such surfaces.

With respect to EMDs or ECDs, these challenges are exacerbated because electronic devices often have a multi-part body which is used to house electronic or other components. Consequently, the smooth contours of the bodies of these devices include various holes, groves, niches, indentations, vents and similar physical features. Such features are typical small, narrow, and difficult to clean, and serve as excellent microorganism reservoirs.

One method of reducing the probability of infection in ECDs is to cover or enclose the device with sanitary or sterile coverings to contain any microbes already present or within a particular ECD to prevent them from contacting a patient or other person who may handle the device. For example, U.S patent application no. 2003/0012371 (“'371”) discloses a cover for a telephone receiver. Although designed to enclose a phone, the '371 invention discloses an open net configuration over the ear and mouth microphones and an open area in the handle portion of the “sock” through which the phone is inserted.

U.S. Pat. No. 8,605,892 (“892”) discloses a protective instrument cover which appears to cover the entire instrument. It teaches a tube having a continuous wall, an open proximal end, a closed distal end, and sealing means operatively associated with the tube. In another embodiment, '892 additionally discloses a continuous wall containing a reservoir formed therein. The disadvantage of the '892 patent is the cover's material is not integrated with any antimicrobial compound. Therefore, although the cover may prevent bacteria from contacting the instrument, the cover itself may be susceptible to bacterial growth and, thereby become its own microorganism reservoir.

Therefore, what is needed is a material that can serve as a cover or “wrap” to substantially cover the outer surfaces of an electronic device which contains an antimicrobial compound, such as silver nanoparticles, along with a method for impregnating such wraps.

### SUMMARY OF THE INVENTION

The present invention is an anti-microbial covering for an electronic device used in a hospital environment. A flexible thermoplastic sheet is impregnated during manufacturing with nano-sized silver particles and wrapped around the electronic device to stop the spread of nosocomial infections. The silver particles vary in shape to maximize the anti-microbial effects of each sheet, and each particle is sized to be less than 60 nanometers in diameter. The present invention includes a process for disrupting nosocomial infections in a healthcare facility by providing an anti-microbial covering to cover electronic devices in the facility. The process includes a method for tailoring the anti-microbial covering to target specific pathogens present in the facility.

Other features and objects and advantages of the present invention will become apparent from a reading of the following description as well as a study of the appended drawings.

### BRIEF DESCRIPTION OF THE DRAWINGS

An anti-microbial covering incorporating the features of the present invention is depicted in the attached drawings which form a portion of the disclosure and wherein:

FIG. 1A is a perspective view of an anti-microbial sheet supported by a folded backing substrate and including perforations and tabs to allow for easy separation of the sheet from the backing substrate;

FIG. 1B is a perspective view of another anti-microbial sheet supported by a folded backing substrate and including concentric perforations;

FIG. 2 a photo micrograph of silver nanoparticles having a rod shape;

FIG. 3 a photo micrograph of silver nanoparticles having an oval shape;

FIG. 4A a photo micrograph of silver nanoparticles having a flower shape;

FIG. 4B a photo micrograph of silver nanoparticles having a prism or triangle shape;

FIG. 5A is a process flow diagram showing an example process to prepare a seed quantity of silver nanoparticles for further use in the process of FIG. 5B;

FIG. 5B is a process flow diagram showing an example process to prepare a quantity of rod shaped silver nanoparticles;

FIG. 6 is a process flow diagram showing a method of making an impregnated anti-microbial sheet;

FIG. 7A is an example of an apparatus for reducing the spread of nosocomial infections using a roller type sheet dispenser;

FIG. 7B is another example of an apparatus for reducing the spread of nosocomial infections using a box type dispenser;

FIG. 8 is an example of an anti-microbial covering in the shape of a bag;

FIG. 9 shows an example of an electronic device (tablet) being covered by an anti-microbial covering incorporating the features of the invention; and,

FIG. 10 is a graph supporting synergistic results against single strain of Gram-positive and Gram-negative bacteria from a study conducted by the inventors to determine the effects of combining silver nanoparticles of different crystal shapes.

### DESCRIPTION OF THE PREFERRED EMBODIMENTS

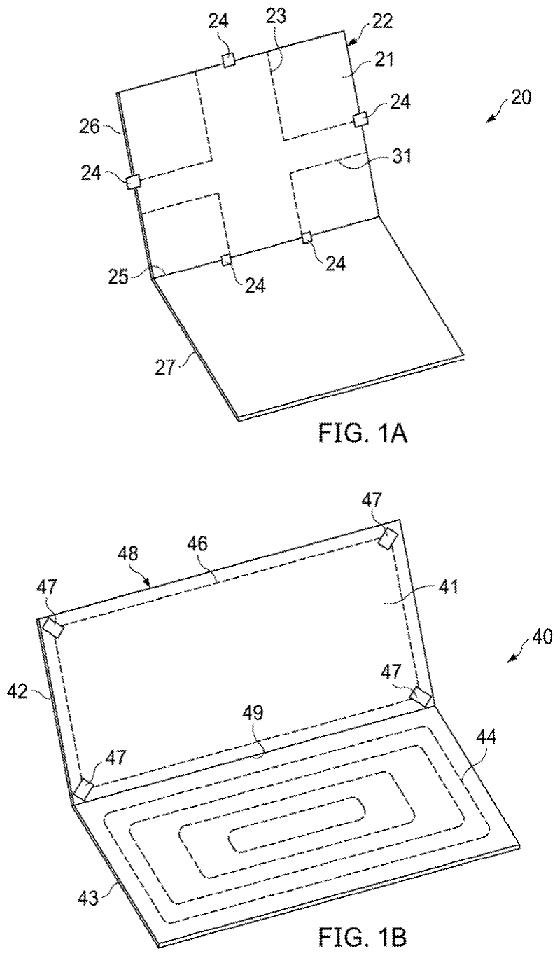

Referring to the drawings for a better understanding of the function and structure of the invention, FIGS. 1A and 1B show example anti-microbial sheets having substrate backings and folded (i.e. configured) to be utilized as a wrap over typical electronic devices used in a hospital environment. The depictions 20,40 each show sheets 20,40 or “wraps” that include a thermoplastic sheet 21,41 impregnated with silver nanoparticles (not shown). Target electronic devices suitable for use in the present invention includes, but are not limited to, smart phones, remote control devices, keyboards, personal computer, tablets, laptop touch-screens, ATM machine monitors, desktop monitors, digital camera screens, GPS navigation screens, mounted touch-screen monitors for factory floors, aviation touch-screen displays, interactive touch-screen displays, interactive white boards (e.g. “smart boards”), iPads®, medical device touch-screens, portable music devices, public display monitors, grocery store self-check-out monitors, touch-screen cash registers, touch-screen coffee machines, touch-screen major appliances, touch-screen media extenders, touch-screen mirrors, touch-screen monitors in vehicles, touch-screen radios, touch-screen soda fountain, touch-screen vending machines, touch-screen tables, touch-screen thermostats, touch-screen televisions, touch-screen voting machines, and touch-screen watches. All of these devices, and more, are utilized in hospital environments. Further, any modern hospital patient room today includes a multiplicity of electronic devices for assisting in the therapy of the patient. For example, patient and treatment rooms can include devices for dispensing medicines, heart monitors, TV control devices, nurse call devices, heart monitors, respiration monitors, and telephones. Moreover, most medical practitioners utilize tablet computing devices that have screens that range in size from 7 to 11 inches to record patient data and issued medical prescriptions.

Each herein described anti-microbial sheet is formed from a base thermoplastic material. The term “thermoplastic,” also known as “thermos softening plastic” is a plastic material, typically comprised of a synthetic plastic polymer, which becomes pliable or moldable above a specific temperature and hardens upon cooling. Most thermoplastics have a high molecular weight. The polymer chains associate through intermolecular forces, which weaken rapidly with increased temperature, yielding a viscous liquid. Thus, thermoplastics may be reshaped by heating and are typically used to produce parts by various polymer processing techniques such as injection molding, compression molding, calendaring, and extrusion. The sheets in present invention are principally formed through sheet or balloon (i.e. blown) extrusion, but various techniques are available for sheet forming of thermoplastics.

Thermoplastic sheets which are suitable for use in the present invention includes, but are not limited to, any light weight or low density polyolefin polymer, preferably low density polyethylene (“LDPE”). Further, as will be described, LDPE may be combined with linear low density polyethylene (“LLDPE”) to allow for increased resiliency. Additives for such thermoplastic may include polyphthalamide (“PPA”) or polybutene (“PBT”), to increase stiffness, elevate softening temperature, and reduce sensitivity to moisture. The inventors are also utilizing polyvinyl chloride also known as polyvinyl or vinyl, and commonly abbreviated “PVC” as a suitable thermoplastic base. Generally, PVC may replace any other disclosed base thermoplastic utilized in any of the herein described sheet formation processes. The same is true for the unitary use of LLDPE as a thermoplastic.

Each thermoplastic sheet can be integrally attached to a substrate 22,48 with an adhesive component (not shown) and contains a plurality of perforations 23, 44, 46, integrated therein such that each sheet is capable of being detached along the perforations utilizing a plurality of tabs 24,47.

Substrates 22,48 suitable for use in the present invention include, but are not limited to, parchment paper, wax paper, polyethylene, polypropylene, polystyrene, polyester, and Glassine, as are known in the industry. Adhesive components suitable for use in the present invention include, but are not limited to, acrylic resin adhesives and the like.

In one preferred embodiment of the present invention, the thermoplastic sheet is constructed of polyethylene such as for example a mixture of the aforementioned LDPE and LLDPE, which is integrated with silver nanoparticles, with each thermoplastic sheet formed to have thickness range of 10 to 1000 microns. Suitable methods of integrating the thermoplastic with silver nanoparticles include, but are not limited to combing particles during extrusion, as will be discussed, and spray coating such particles onto the hardened sheet exterior after cooling. Extrusion forming of the present invention is preferred because during usage a sheet may be deformed during an encapsulation process of an electronic device or simply through usage in and around a hospital environment. Since an extrusion process impregnates the silver nanoparticles throughout the thermoplastic material, a deformation event will not diminish the effectiveness of the antimicrobial process of the silver nanoparticles since a deformation event would only uncover new particles to react with microorganisms. In addition, an extrusion process inherently makes available silver nanoparticles to combat microorganisms on all sides of a thermoplastic sheet. So, for example, a bag holding an electronic device will kill microorganisms on both the inside and outside of the bag. Finally, extrusion is preferred because a thermoplastic sheet with particle impregnation does not lesson in effectiveness with wear, but may instead refresh its surface toxicity to microorganisms as new amounts of silver nanoparticles are exposed on the surface of the sheet with abrasion. A simple surface coating silver nanoparticles does not exhibit any of these qualities.

Referring to FIG. 1A, the thermoplastic sheet 21 is integrally attached to a substrate 22 with an adhesive component (not shown) and contains a plurality of perforations 31 formed therein such that the thermoplastic sheet 21 is capable of being detached along the perforations 31 utilizing a plurality of tabs 24 formed within each thermoplastic sheet 21. The substrate 22 can be bifurcated along a center fold 25 thereby creating a front substrate 26 above the center fold 25 and a back substrate 27 below the fold 25, the perforations 31 being positioned on the front substrate 26 such that a cross-like shape 23 is formed.

For illustration purposes, an electronic device, such as a cell phone, may be placed at the center of the cross shape formed by the perforations 31. The portion of the sheet 21 inside the perforations 31 is lifted from the substrate 22 via tabs 24 and the sheet wrapped over an electronic device, with each arm of the cross shaped portion being folded over the device to cover all portions of its outer surface. The removed portion of the sheet will cling to the outer surface of the device through static attraction, or from any residual adhesive remaining from the substrate 22.

Similarly, FIG. 1B shows another thermoplastic sheet 41 integrally attached to a substrate 48 with an adhesive component (not shown) and contains a plurality of perforations 44,46 integrated therein such that the thermoplastic sheet 41 is capable of being detached along such perforations utilizing a plurality of tabs 47. Substrate 48 may be bifurcated along a center fold 49, thereby creating a front substrate 42 above the center fold 49 and a back substrate 43 below the center fold 49. As may be seen, the perforations 44 supported on the back substrate 43 are configured into concentric shapes so that a user may separate the sheet 41 from the substrate 43 to accommodate various sized devices by selecting and lifting the sheet at an appropriate perforation perimeter. The sheets on the back substrate 43 are shown without lifting tabs, and may be separated from substrate 43 by simple manual manipulation of the combined sheet and substrate. While rectangles with eased corners are shown, it will be understood that any shapes, whether concentric or not, may be formed in the sheets using common die-cutting methods. In operation, a device may be placed on one of the outlined perforation shapes on substrate 43 and a user lifts the appropriate sheet shape in order to cover the entire surface of the device.

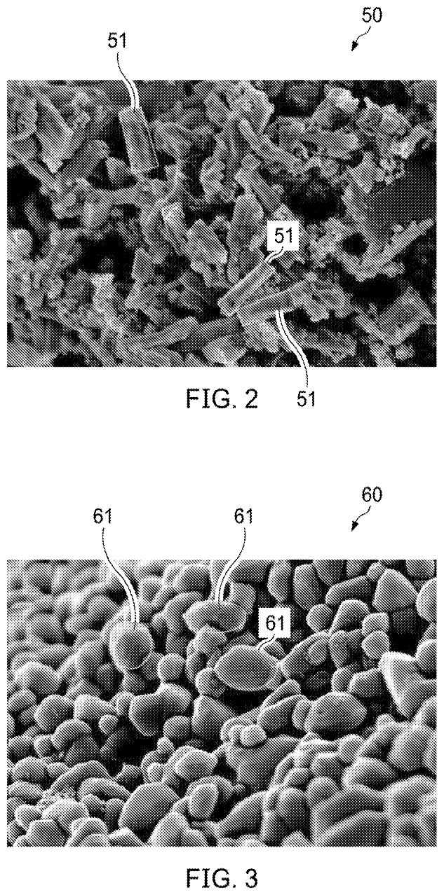

Referring now to FIGS. 2, 3, and 4A-4B, a series of electron micrographs are presented showing the various shapes of silver nanocrystals forming the basis of nanoparticles for impregnation into the present anti-microbial sheets. “Silver nanoparticles” are nanoparticles of silver of between 1 nm and 100 nm in size. Variations in shapes of nanoparticles in general are known to affect the chemical properties of different nanoparticle based substances. Silver nanoparticles have an extremely large surface area and produce a high quantity of “ligands,” ions or functional molecular groups, which bind to a central metal atom and form a coordination complex. The inventors noticed that since silver in nano-sized particle solutions are more toxic to bacteria than larger sized colloidal particle solutions, varying the shapes of those nano-sized particles would likely further enhance the antimicrobial efficacy of such a silver nanoparticle solution. Their idea was that use of a single geometric shape of silver nanoparticles causes spatial gaps or interstices between each silver particle, which lesson the potential toxicity of the particles. This gap concept applies to all unitary geometric shapes of silver nanoparticles. Hence, varying the particle structure of precipitated silver during crystal synthesis might result in enhanced anti-microbial effects.

One explanation for this potential effect is that each shape has a different surface to volume ratio and thus each has different high-atom-density facets. These facets act as maximum reactivity sites leading to varying strength in antibacterial activity against bacteria. Based on different cell wall composition of bacteria, Gram-positive and Gram-negative bacteria respond differently to specific shapes. Under this theory, silver nano-rods and nano-wires might be more effective against Gram-positive bacteria, whereas silver nano-prisms (i.e. nano-triangles) might be more effective against Gram-negative bacteria as antimicrobial agents. By combining different particle shapes into a single preparation the total antimicrobial effect of a quantity of silver nanoparticles as an impregnation agent might be increased, not only because the overall density of silver is increased, but also because of the enhancement of toxicity over a range of microorganisms. Hence, in order to enhance further the antimicrobial effects of the above described silver nanoparticles the inventors conceptualized the idea of combining various crystal shapes of silver nanoparticles into a single preparation material to improve their antimicrobial properties.

To confirm this theory, the inventors performed antibacterial studies using varying combinations of different shapes, eventually arriving at the most efficient antibacterial combination with no gaps in the lattice structure as verified through SEM/AFM imagery. Tables 1-3 below shows testing efforts over different concentrations of silver nanoparticles and varying crystal shapes. This testing resulted in a preferred formulation F3 shown in bold in Table 3 which exhibited the best broadscale antimicrobial effects. In fact, in comparing those results to expected results, the inventors concluded that antimicrobial effects were at least additive in efficacy with the combination of different crystal shapes and in some instances superior to simple additive effects. More importantly, the antimicrobial efficacy over a spectrum of microorganisms is increased over what a single crystal shape can achieve because each shape interacts differently with different microorganisms. The inventors' research indicates that certain shaped silver nano-particles appear to be more toxic to certain types of microorganisms. The inventors theorize that this is because ligand availability varies chemically in accordance with a potential chemical matching between a microorganism protein coating and the shape or availability of a nearby silver nanoparticle ligand. For example, a prism shaped nanoparticle may fit (i.e. react) better with the coating of a S. aureus bacteria, but a sphere shaped nanoparticle may react more toxically with E. coli. Hence, the combination of the two shapes provides a synergistic effect because it not only provides an additive effect of toxicity by simply increasing the strength of a nanoparticle solution for a particular volume, but it also provides a synergistic effect by broadening the effectiveness of the antimicrobial effect against two or more microorganisms. Prior silver coating preparations did not exploit this strategy to provide a broad spectrum of microorganism toxicity.

TABLE 1

| Sr. | | | Original |
|---|---|---|---|
| No. | Formulation type | Code | Conc. (ppm) |
| 1 | Spherical silver | SSN | 0.5 |
| | nanoparticles | | |
| 2 | Nanorods | NR | 0.56 |
| 3 | Nanoprisms | NP | 0.38 |
| 4 | Nanoprism- scale up | NP-L | 2 |
| 5 | Nanowires | NW | 1.57 |

TABLE 2

| | | Combinations Tried | | | | | |
|---|---|---|---|---|---|---|---|
| | Nano- | F1 | F2 | F3 | F4 | F5 | F6 |
| Sr. | particle | Ratio | Ratio | Ratio | Ratio | Ratio | Ratio |
| No. | Type | (ppm) | (ppm) | (ppm) | (ppm) | (ppm) | (ppm) |
| 1 | SSN | 1 | 2 | 1 | 1 | 1 | 1 |
| 2 | NR | 1 | 1 | 2 | 1 | 1 | 1 |
| 3 | NP | 1 | 1 | 1 | 2 | 1 | 1 |
| 4 | NP-L | 1 | 1 | 1 | 1 | 2 | 1 |
| 5 | NW | 1 | 1 | 1 | 1 | 1 | 2 |

TABLE 3

| Sr. | | NCIM | F1 | | F2 | | F3 | | F4 | | F5 | | F6 | |
|---|---|---|---|---|---|---|---|---|---|---|---|---|---|---|
| No. | Organism | No. | MIC | MBC | MIC | MBC | MIC | MBC | MIC | MBC | MIC | MBC | MIC | MBC |
| 1 | E. coli | 2065 | 0.0375 | 0.075 | 0.1425 | 0.285 | 0.02875 | 0.0575 | 0.07375 | 0.1475 | 0.07 | 0.14 | 0.03 | 0.06 |
| 2 | E. cloaceae | 2015 | 0.075 | 0.15 | 0.1425 | 0.285 | 0.0575 | 0.115 | 0.036875 | 0.07375 | 0.07 | 0.14 | 0.06 | 0.12 |
| 3 | P. mirabilis | 2241 | 0.0375 | 0.075 | 0.1425 | 0.285 | 0.02875 | 0.0575 | 0.07375 | 0.1475 | 0.07 | 0.14 | 0.03 | 0.06 |
| 4 | S. aureus | 2079 | 0.0375 | 0.075 | 0.1425 | 0.285 | 0.02875 | 0.0575 | 0.07375 | 0.1475 | 0.07 | 0.14 | 0.03 | 0.06 |
| 5 | S. marscences | 2078 | 0.15 | 0.3 | 0.1425 | 0.285 | 0.02875 | 0.0575 | 0.07375 | 0.1475 | 0.07 | 0.14 | 0.03 | 0.06 |
| 6 | P. aeruginosa | 2036 | 0.0375 | 0.075 | 0.017812 | 0.035625 | 0.02875 | 0.0575 | 0.018437 | 0.036875 | 0.035 | 0.07 | 0.06 | 0.12 |
| 7 | S. pneumoniae | Lab | 0.15 | 0.3 | 0.1425 | 0.285 | 0.02875 | 0.0575 | 0.07375 | 0.1475 | 0.07 | 0.14 | 0.03 | 0.06 |
| | | strain | | | | | | | | | | | | |
| 8 | S. aureus | Clinical | 0.0375 | 0.075 | 0.1425 | 0.285 | 0.0575 | 0.115 | 0.036875 | 0.07375 | 0.035 | 0.07 | 0.03 | 0.06 |
| | | isolate | | | | | | | | | | | | |

Pursuant to Tables 1-2, a series of formulations were organized for testing F1-F6 that vary the parts per million (“ppm”) by concentration volume ratio for each silver nanoparticle crystal shape as indicated. While the original ppm concentrations varied for the individual shapes as shown in Table 1 (right most column), the concentrations were normalized to approximately 0.50 ppm for combinatorial testing with a guaranteed range of between 0.46 ppm and 0.60 ppm. This range limitation provides assurance that testing results will not be skewed in antimicrobial effect when tested against a broad range of microorganisms.

Testing results shown in Table 3 list a number of bacterial strains against which various silver nano-particle formulations (Sr. Nos.) were tested. For each tested formulation F1-F6, a minimum inhibitor concentration (“MIC”) and a minimum bactericidal concentration (“MBC”) was elucidated for each combination. The object of the test was to select an optimal formulation prior to combining the silver nanoparticles with a thermoplastic base medium for sheet extrusion As expected, various shaped silver nanoparticle crystals had varying toxicity results for different types of microorganisms—in this case strains of bacteria. However, the formulation F3 had the broadest efficacy across the eight (8) strains and was selected for product extrusion. Hence, formulation F3 is currently the preferred silver nanoparticle crystal shape combination.

Another study was also done to test for synergistic toxicity effects. As indicated above, the inventors had already observed a synergistic effect related to the ability for a combination of different silver nanoparticle crystal shapes to target different types of microorganisms as a group by exhibiting a higher level of toxicity between matched toxic susceptibility based on crystal shape to a type of microorganism. However, a different type of synergy would also be desirable—an effective toxicity beyond expected additive toxicity expectations when combining two or more silver nanoparticle crystal shapes with a single microorganism, irrespective of matched toxic susceptibility.

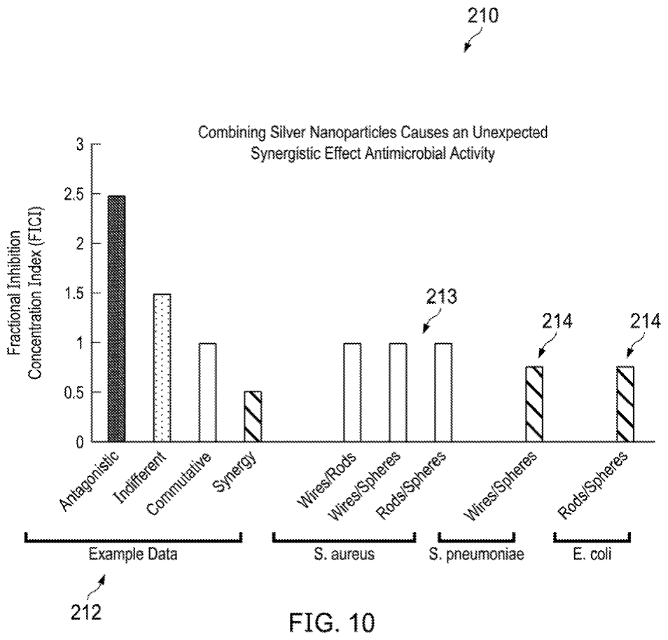

FIG. 10 displays a graph 210 that shows synergistic effects against single strain of Gram-positive and Gram-negative bacteria using various shapes of silver nano-crystals. The graph 210 represents the results of a study conducted by the inventors to determine the effects of combining silver nanoparticles of different crystal shapes (“SNPx”) on their antimicrobial activity against representative Gram-positive and Gram-negative bacterial pathogens. The silver nanoparticles crystal shapes comprised nanowires (“Wires”), nanorods (“Rods”), and nanospheres (“Spheres”) tested against Staphylococcus aureus (Gram-positive), Streptococcus pneumoniae (Gram-positive), and Escherichia coli (Gram-negative) in one technical replicate assay.

The study employed a modified “checkerboard assay” strategy to test the inhibitory potential of the three different silver nanoparticles crystal shape combinations over a range of concentrations. A checkerboard assay is a well known testing technique to determine if the combined effects of a plurality of substances provides a targeted result greater than an expected result. The checkerboard assay setup used in this study simultaneously determined the minimum inhibitory concentration or “MIC” for each individual compound alone and the fractional inhibitory concentration or “FIC” for each silver nanoparticles crystal shape when tested in combination with another silver nanoparticles crystal shape. The data was then used to determine a FIC Index (“FICI”) for each combination using the following formula:

FICI=FIC_{SNP1}+FIC_{SNP}2

where

FIC_{SNP1}=MIC_{SNP1}

in combination/MIC_{SNP1} alone, and

FIC_{SNP2}=MIC_{SNP2} in combination/MIC_{SNP2} alone

The checkerboard assay was modified to simultaneously determine the MIC and FIC for each compound in the pair on the same well plate, so that the resulting data can be used to calculate a FICI for each pair, and such that synergistic effect would be detected if a FICI value <1; a commutative effect would be detected if the FICI value =1; an indifferent effect would be detected if 1<FICI≤2; and an antagonistic effect would be detected if the FICI value >2. The “Example Data” portion 212 in FIG. 10 provides a key guide to the table. In the study, a FICI value is only reported where both the MIC and FIC could be determined simultaneously in the Checkerboard Assay. The data presented in FIG. 10 strongly suggests that different combinations of the three silver nanoparticles crystal shapes tested produce either commutative effects 213 or synergistic effects 214. The observations of synergistic effects 214 is highly unexpected considering that the silver nanoparticles crystal shapes are likely inhibiting growth via the same mechanism. In addition, the data supports the theory by the inventors that the combination of at least three or more different silver nanoparticle crystal shapes achieves maximal coverage to inhibit growth in a range of Gram-positive and Gram-negative microorganisms typically present in a healthcare environment. The full study results are included as a supporting appendix to this application.

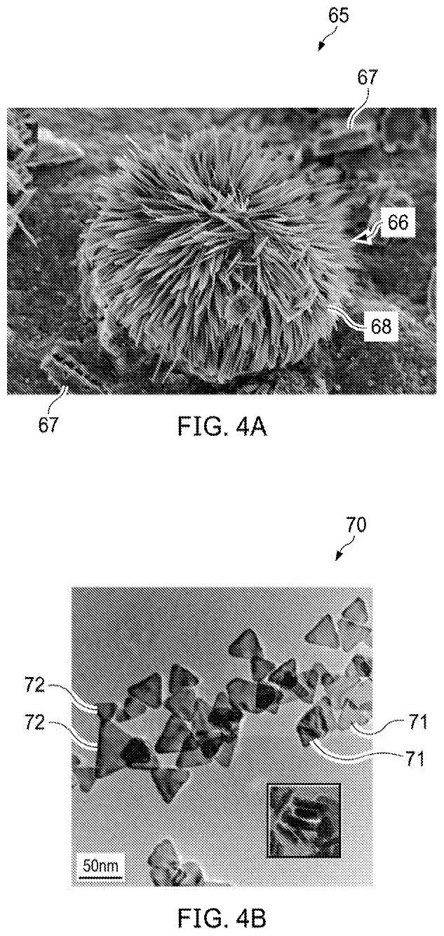

Therefore, based on the above described testing results, the inventors have concluded that by varying combinations of silver nanoparticle crystal shapes, and by varying the percentage by weight of each shape relative to other shapes, the inventors are able to optimize a formulation of silver nanoparticles for impregnation into a thermoplastic sheet that maximizes an antimicrobial effect in the sheet. Moreover, a formulation may be created in which a quantity of one shape of crystal may be increased so that interstices observed via electro-micrograph (e.g. FIG. 4B) between two or more other crystal shapes may be filled, essentially optimizing the formulation to minimize spatial interstices between each crystal. Such formulation flexibility allows for various types of formulations to be created to optimize an impregnated thermoplastic sheet to resist or be more effective against a particular type of bacteria or virus. A formulation may be designed therefore, after testing, to target a particular pandemic threat, such as for example the Covid-19 virus or seasonal influenza viruses.

As indicated above, the inventors discovered that varying the shapes of silver particles, along with control of their sizes and concentrations, has a strong effect on antimicrobial efficacy. Hence, control and preparation of various shapes and sizes of silver nanoparticles is necessary to achieve a desired antimicrobial outcome. By using various chemical techniques, various silver nanoparticle shapes may be realized, such as for example the following shapes: rod shaped, wire shaped, sphere shaped, oval or ellipsoid shaped, triangle or prism shaped, and flower shaped.

The most common methods for producing silver nanoparticles fall under the category of wet chemistry, or the nucleation of particles within a solution. This nucleation occurs when a silver ion complex, usually AgNO₃ or AgClO₄, is reduced to colloidal silver in the presence of a reducing agent. When the concentration increases enough, dissolved metallic silver ions bind together to form a stable surface. The surface is energetically unfavorable when the cluster is small, because the energy gained by decreasing the concentration of dissolved particles is not as high as the energy lost from creating a new surface. When the cluster reaches a certain size, known as the critical radius, it becomes energetically favorable, and thus stable enough to continue to grow. This nucleus then remains in the system and grows as more silver atoms diffuse through the solution and attach to the surface. When the dissolved concentration of atomic silver decreases enough, it is no longer possible for enough atoms to bind together to form a stable nucleus. At this nucleation threshold, new nanoparticles stop being formed, and the remaining dissolved silver is absorbed by diffusion into the growing nanoparticles in the solution. Varying the rate and density of ions through various chemical agents and ambient conditions allows for the shape of the particles to be determined. For example, the attachment of a stabilizing agent will slow and eventually stop the growth of silver particles. A common capping agent is trisodium citrate and polyvinylpyrrolidone (“PVP”), but others may be used to varying conditions and to control particle size and shape of the silver particles, along with surface properties. Some methods of producing various particle shapes are described below, but a thorough explanation of various chemical techniques used to shape and size silver nanoparticles, and the underlying science, is omitted since such detail is not necessary for a complete and full understanding of the herein described invention.

FIG. 2 shows an electronic micrograph 50 of a grouping of silver particles having generally a rod like shape 51 and having a diameter of less than 60 nanometers.

FIG. 3 shows an electronic micrograph 60 of a grouping of silver particles having generally an oval shape 61 and having a diameter of less than 60 nanometers.

FIG. 4A shows an electronic micrograph 65 of a single silver particle 66 in a varied grouping of different shaped silver particles, such as rods 67. The particle 66 has a generally flowered shape with many particle extensions or “arms” 68 emanating from a central index point. Each arm has a diameter of less than 10 nanometers.

FIG. 4B shows an electronic micrograph 70 of a plurality of silver particles having generally a triangle or “prism” shape 71 in a varied grouping of different sized triangle shaped particles. The triangle shaped particles vary in size 72 of between 20 and 60 nanometers.

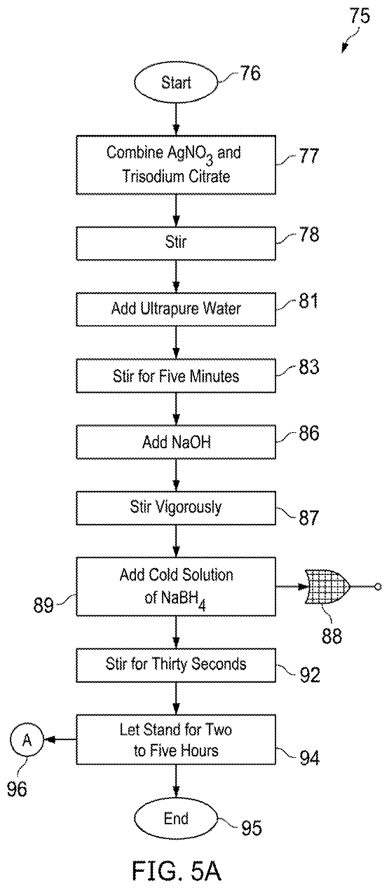

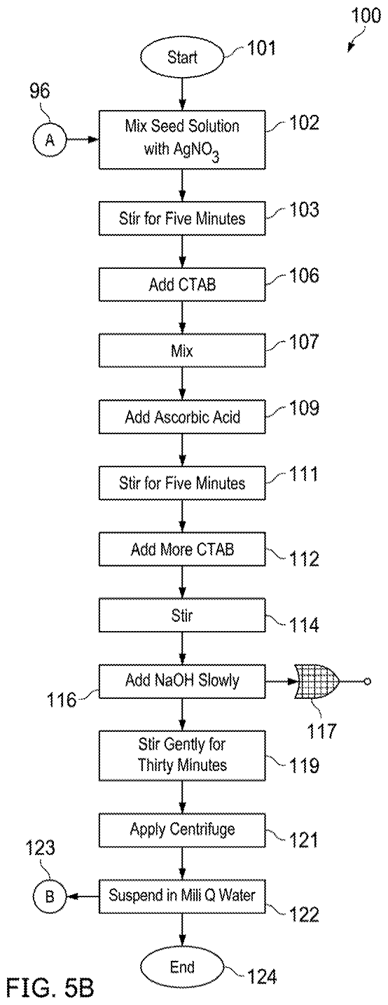

FIGS. 5A-5B show an example process for synthesizing the rod shaped particles shown in FIG. 2. Referring to FIG. 5A, initially a series of chemical steps 75 are taken to produce a “seed” solution of silver particles in a workable volume. As shown, after the start 76, 20 mL of 2.5 mM AgNO₃ solution is combined with 20 mL of 2.5 mM trisodium citrate 77 and stirred 78. One hundred fifty mL of Ultra-pure (e.g. Milli-Q) water is added 81 to the mixture of 77 and stirred for 5 minutes 83. Six mL of 10 mM NaOH solution is added dropwise 86 and stirred vigorously 87, and then 6 mL of ice cold solution of 10 mM NaBH₄ is combined 89 with the solution of 86 while stirring vigorously. The colorless solution should then turn yellow 88 and the yellow solution should be continued to be stirred for 30 seconds 92 and then let stand 94. A seed solution (A) 96 should be available to be use between 2-5 hours after completion of the seed process of 75 and may be used to form specific shapes and sizes of silver nanoparticles with further chemical processing as will be described. This ends 95 seed solution process 75.

Referring now to FIG. 5B, process 100 discloses a method to produce rod-shaped silver nanoparticles using the seed solution formed in process 75. As shown, process 100 starts 101 with the step of 22.5 mL of the seed solution (A) 96 being mixed with 22.5 mL of 10 mM AgNo₃ 102 and stirred well for 5 minutes 103. Twenty mL of 80 mM CTAB (cetyl trimethylammonium bromide) solution is added 106 and mixed well 107. After this, 45.02 mL of 100 mM ascorbic acid solution is added 109 to the mixture of 106 and stirred well for 5 minutes 111. The solution is then enlarged by adding 112 880 mL of 80 mM CTAB with continuous stirring 114. Finally, 9 mL of 1M NaOH solution is added slowly 116 to the above mixture and the solution should turn a yellowish to red color 117. The solution is then continuously stirred gently for an additional 30 minutes 119. The above produces nano rods in solution and a centrifuge is used 121 to separate out the nano-rods. A centrifuge run at 11,000 RPM is typically suitable for such separation. The resulting separated nano rods are left suspended in ultra-pure water 122 until needed for impregnation with a thermoplastic (B) 123 in an extrusion process.

Seed solution A 96 may be further chemically processed to create other shapes and sizes of silver nanoparticles, such as sphere shaped nanoparticles and wire shaped nanoparticles. For example, to produce wire shaped silver nanoparticles, the same process may be used as for forming rod shaped nano particles except that 155.65 mL of silver seed solution is used instead of 22.5 mL in step 102, and 750 mL of 80 mM CTAB is used instead of 880 mL in step 106 of process 100. All other steps are identical in process 100.

To form prism shaped silver nanoparticles, the following steps are satisfactory. As will be observed, no seed solution is utilized in making prism shaped nanoparticles:
Step 1. Combine 885 mL of 0.1 mM AgNo₃ in a beaker and stir.
Step 2. Add 53.4 mL of 30 mM trisodium citrate to the above solution via a dropwise process.
Step 3. Add 53.4 mL of 0.7 mM Polyvinyl Pyrilidone via a dropwise process to above mixture formed in step 2.
Step 4. Add 30% by weight (i.e 2.12 mL) of hydrogen peroxide to the above mixture resulting from step 3 immediately after completion of step 3.
Step 5. Add 884.01 mL of 100 mM sodium borohydrite dropwise to above mixture formed in step 4 and continue stirring.
Step 6. Continue stirring the mixture formed in step 5 for 30 minutes until the solution changes to a brownish red color. A centrifuge may then be utilized on the mixture to separate out the prism nanoparticles.

Sphere shaped silver nanoparticles are formed during the seed production procedure 75 shown in FIG. 5A and hence no additional processing is required except to apply a centrifuge to the seed solution to extract the sphere shaped silver nanoparticles.

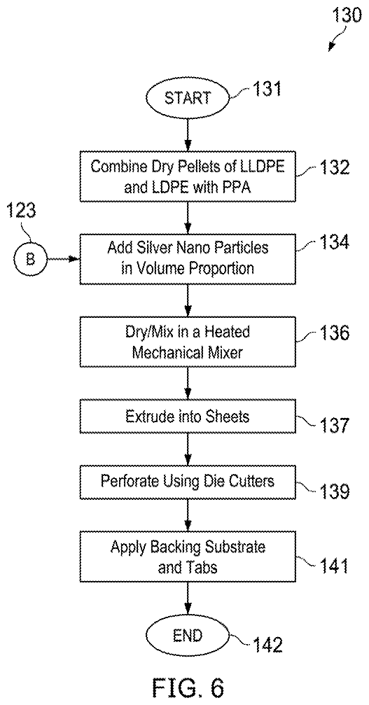

Referring to FIG. 6, process 130 is a suitable process for making an antimicrobial sheet. The process starts 131 and pellets of LLDPE and LDPE are combined 132 in a ratio of approximately 80% LDPE and 20% LLDPE by weight. However, a preferred method to make a batch of approximately 10 kg of antimicrobial sheets includes combining of the polyethylene pellets with other additives in dry quantities pursuant to the following component amounts by weight:

| | | | |
|---|---|---|---|
| 1. | LLDPE pellets: | 8 | kg |
| 2. | LDPE pellets: | 2 | kg |
| 3. | Polyphthal amide: | 150 | gm |
| 4. | UV protection agents: | 200 | gm |
| 5. | Plastic brightener: | 100 | gm |
| 6. | Antistatic agent: | 100 | gm |
| | Total | 10,550 | gm |
| 7. | Silver nanoparticles: | 900 | microgram |
| | Total | 10,550.0009 | gm |

The resultant weight percentages are listed below in Table 4:

TABLE 4

| No. | Component | Weight Percentage |
|---|---|---|
| 1 | LLDPE | 75.82 |
| 2 | LDPE | 18.95 |
| 3 | Polyphthalamide | 1.42 |
| 4 | UV protection agents | 1.89 |
| 5 | Plastic brightener | 0.94 |
| 6 | Antistatic agent | 0.94 |
| 7 | Silver nanoparticles | 0.000009 |

Items 1-6 are combined via dry mixing 132 in a tray or other suitable vessel. The silver nanoparticles (B) 123 from the processes (75,100) in FIGS. 5A and 5B are then combined with the plastic ingredients 134 and dry mixed 136 in the presence of polyethylene glycol to facilitate the removal of water. The silver nanoparticles that are added may include one or more shapes and sizes produced in the above described processes. A preferred concentration ratio of shapes and sizes of silver nanoparticles are 2:1:1:1 for the following shapes, respectively: rods (2); prisms (1); spheres (1); and wires (1). Each of the aforementioned particle shapes may have varying concentrations resulting from the above formation steps. However, in order to meet the above concentration ratios concentrations of each shape must be known and normalized with respect to the concentrations of the other shapes in order to properly combine all of the shapes pursuant to the above stated concentration ratio. So, for example, if a supply source for each of the above preferred shapes was available at a concentration level of 1.0 microgram per mL, and a desired total volume of 5 mL of silver nanoparticles was desired, then in order to meet the above preferred combination ratio, the following quantities shown in Table 5 would be needed.

TABLE 5

| Concentration | | Source | No. of mL |
|---|---|---|---|
| Ratio | Shape | Concentration | Required |
| 2 | Rods | 1.0 μg/mL | 2 |
| 1 | Prisms | 1.0 μg/mL | 1 |
| 1 | Spheres | 1.0 μg/mL | 1 |
| 1 | Wires | 1.0 μg/mL | 1 |
| | | | Total Volume = 5 mL |

Also, size limitations must be maintained. All silver nanoparticles should be less than or equal to 60 nanometers at their widest diameter, with a preferred range of between 10 and 50 nanometers. While a quantity of 900 micrograms is utilized in the preferred present method for the disclosed quantity, the inventors have seen satisfactory results using a range 500 micrograms to 50 mg of silver nanoparticles in such a process.

The combination of silver nanoparticles with the other thermoplastic ingredients are dry-mixed in a heated mechanical mixer 136 at approximately 300-500 RPM, a temperature of 70°-90° C., and a time duration of 30 minutes. This removes moisture from the silver nanoparticles and thoroughly mixes and impregnates the silver nanoparticles (via absorption) into the thermoplastic. Sheet or balloon extrusion then occurs at 250° C. to produce a thin sheet of silver nanoparticle impregnated thermoplastic 137. The resulting antimicrobial sheet has superior antimicrobial characteristic, is highly flexible, resilient, and transparent. Additional additives may be included to add color to the formed antimicrobial sheets, or increase optical light scattering so that the sheets are translucent. A suitable sheet thickness for the herein described invention is any thickness less than 100 microns. However, by decreasing thickness further, the surface contact area of silver nanoparticles with microorganisms, such as bacteria, increases thereby increasing antimicrobial properties. Hence, a preferred thickness is 30 microns where the tensile strength is sufficient to cover devices such as medical instruments while withstanding regular use by medical personnel. Nevertheless, sheets having a thickness of 10-30 microns are possible and would be satisfactory for many medical environments.

In one form, the herein described sheets may be dispensed in convenient rolls of non-adhesive applied sheets without backing sheets holding a separate adhesive layer. For those sheets without a separate adhesive layer, or for sheets not having self-closing tabs or bags with self-sealing mechanisms, the sheets must surround and adhere to the exterior of the targeted electronic device, such as EMDs and ECDs, and/or adhere to itself by wrapping the targeted device until surrounded. For this type of embodiment, an important functional property that preferably should be exhibited by an extruded sheet is a “cling” force property sufficient for it to adhere satisfactorily to itself and other targeted surfaces, such as the exterior to an electronic device like a cell phone or tablet. Thermoplastic resin films do not generally possess inherent cling characteristics but must be obtained through the use of so-called cling agents or adhesives within the base sheet. Adhesives are chosen for their ability to produce a surface on a thermoplastic sheet (or film) that can be sealed, opened and resealed, and are selected with due care in consideration of the use of thermoplastic film. Generally, the adhesives are referred to in the industry as “tackifiers” since they make the surface of the film “tacky” or sticky. Examples of preferred tackifiers include mixtures of rosin esters and styrene-isoprene-styrene block copolymers. Whatever tackifiers are utilized, they must include an effective amount to provide sufficient cling to different substrates such as, for example, glass, plastic, ceramic, stainless steel, laminated cardboard, and aluminum. Tackifiers are mixed with the thermoplastic sheet base such as, for example, polyolefins like LDPE and LLDPE during the sheet extrusion process and may include one or more of the following: a resin such as ethylene vinyl acetate 40 (“EVA”), a resin of ethylene methyl acrylate (“EMA”), or styrene-isoprenestyrene (SIS) block copolymer in combination with a rosin ester tackifier. Examples of preferred rosin ester tackifiers are Sylvaros™ PR R85 and Sylvaros™ PR 295 which are available from Arizona Chemical located in Panama City, Fla. As is known, SIS and rosin ester tackifiers may be combined with a base polyolefin, in varying weight proportions, to cause a desired level of tackiness.

The addition of a tackifier provides the cling property necessary for a thermoplastic sheet to surround and stick to a computing or electronic device. However, given the frequent handling of typical electronic devices used in a healthcare environment, a minimum degree of cling property must be exhibited by the sheet to sustain an effective covering of the electronic device during daily use. The strength of the cling property is referred to in the industry as the “cling force” exhibited by the sheet or film, and is measured by various ASTM tests such as ASTM D4649 or ASTM D5458. A typical cling force of a plastic film to cling to the same or a similar film should be approximately between 300 and 500 grams as measured in accordance with ASTM D4649. This also approximates the necessary cling force to a targeted device surface, assuming a clean plastic surface. At a minimum, a cling force of approximately 75 grams as measure by ASTM D5458 should be maintained to ensure non-release of the film to surround a targeted device. Further information regarding the industry techniques and additives used to achieve a suitable cling force for a plastic film, or the testing of such cling forces is omitted as these techniques are understood and not necessary for a complete understanding of the herein described invention. In addition, U.S. Pat. No. 6,692,805 issued to Bonke in 2004 provides further information regarding the state of the art in cling forces and achievement thereof, and which is herein incorporated by reference in its entirety.

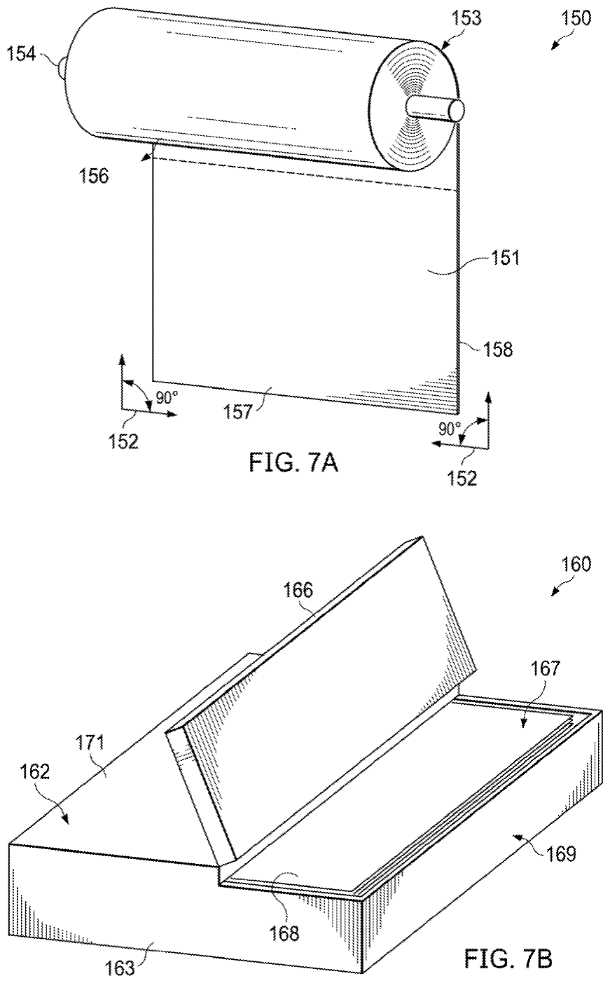

Once extruded and cooled, a pattern of perforations, or shaped perforations, may be made using die cutters 139. A backing substrate along with tabs may also be added 141 using industry known techniques. Manufactured sheets may be cut into individual sheets and dispensed, or placed on a roller for dispensing by tearing along perforations. For example, FIG. 7A shows a paper towel type dispenser 150 holding a roll 153 of antimicrobial sheets suspended by a roller 154 and positioned in conveniently accessed location in a hospital or clinic environment. Each antimicrobial sheet is rectangular in shape having right angle corners 152 and may include a backing substrate 158 so that a user may grasp the sheet 151 from its lower edge 157 and separate it from roll 153 via perforations 156. They may then remove the antimicrobial sheet using manual manipulation or place an electronic device upon the upper surface of the sheet and lift the sheet from its backing to cover the entire surface of the electronic device, as described above in the description for FIGS. 1A-1B.

It is acknowledged that a standard for a minimum level of efficacy against microorganisms is necessary for the current invention. Hence, a minimum inhibitory concentration or MIC of silver nanoparticles solution may be tested against a targeted specific microorganism in a laboratory applicable to a present healthcare facility. Testing may be done with silver nanoparticles in solution against such a targeted microorganism to establish minimum concentration level of silver nanoparticles having varying shapes and sizes. Using those results, a standard of 10 times the minimum effective concentration level may be established as a MIC per square meter of area of a produced antimicrobial sheet. Hence, any organization can target and establish a MIC for its antimicrobial sheets tailored to be used in their medical operations, and a manufacturer can produce and supply such sheets meeting those established minimums. For the process 130 disclosed, a broad MIC of 0.02 to 2 microgram per mL is preferred to achieve a broad spectrum of antimicrobial efficacy in the described antimicrobial sheets produced. In addition, as mentioned previously, tailored formulations may be created to maximize a toxicity effect against a targeted pathogen by varying proportions of volumes of a selected shape of silver nanoparticles known to have a higher degree of toxicity against the target.

FIG. 7B shows a dispensing box 160 holding a set of stacked, pre-separated sheets 168 accessible via opening 167. A door or lid 166 is hinged from rear upper surface 171 and biased downward to seal the opening 167 and keep dust or debris from settling on the sheets or inside the dispensing box. Opening 167 is defined by the upper edges of front upstanding portion 169 and a pair of lateral upstanding side panels 163. The dimensions of the dispensing box determine how many sheets may be stored within it. As may be understood, rear portion 162 may be affixed in a conventional manner to a wall or stand so that the dispenser 160 may be positioned in convenient locations within a hospital or clinic in order for medical personnel to easily access the antimicrobial sheets. A companion platform may also be provided adjacent to the dispensing box (not shown) in order to facilitate the wrapping of the antimicrobial sheet around an electronic device.

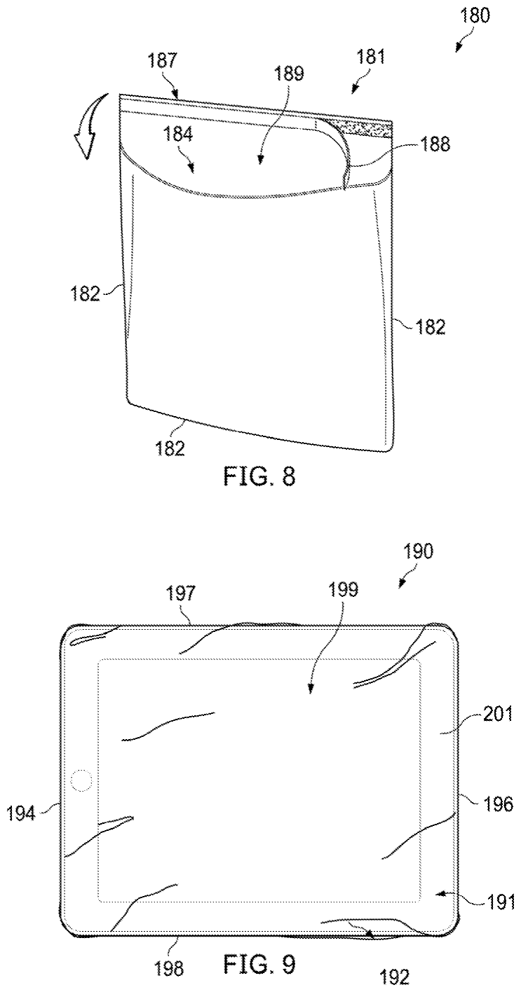

Another convenient shape made from antimicrobial sheets that addresses nosocomial infections is an antimicrobial bag. FIG. 8 shows such a bag 180 that is formed by thermo-sealing a bi-folded antimicrobial sheet at edges 182 to form an opening 184. An elongated edge 181 includes a resealing edge 187 holding an adhesive strip 188, or similar sealing device. Rather than peal and wrap an antimicrobial sheet around an electronic device, a device may simply be placed through opening 184 in direction 189 and the top edge of the bag turned over to engage a lower portion of the bag to seal it. A re-sealable zipper type edge, similar to that used in Ziploc® container bags, may also be used to seal each bag resulting in a bag-type antimicrobial container. Such bag-type antimicrobial containers may be more practical in some environments, especially where response time is limited as in a hospital emergency room. Further, a bag-type antimicrobial container may be simply more convenient for workers to access and utilize. Antimicrobial sheets used to make such a bag are slightly thicker than a nominal single sheet, optimally 40 to 60 microns in thickness, and would vary in size to form bags suitable for enclosing electronic devices of varying sizes and shapes.

Referring now to FIG. 9, an antimicrobial sheet 192 has been placed over an electronic tablet 191 covering around all edges 194, 196, 197, and 198, and including margin area 201 surrounding screen 199. The antimicrobial sheet exhibits transparent optical properties so that the screen 199 of the tablet 191 may be freely viewed by a user through sheet 192. Further, the inherent thin, flexible nature of any antimicrobial sheet allows for a user to access all buttons or levers necessary on the device without interference with the haptics of the touchscreen. This allows for the normal operation of the tablet by a medical practitioner. Hence, the combination 190 allows for the full functionality of a tablet for hospital use, while interrupting the spread of communicative microorganisms that cause nosocomial infection.

While I have shown my invention in one form, it will be obvious to those skilled in the art that it is not so limited but is susceptible of various changes and modifications without departing from the spirit thereof.

Having set forth the nature of the invention, what is claimed is:

1. An anti-microbial covering for an electronic device used in a hospital environment, comprising:
a. a flexible thermoplastic sheet adapted to surround said electronic device;
b. wherein said thermoplastic sheet comprises a mixture of low density polyethylene, linear low density polyethylene, and polyphthalamide;
c. wherein said thermoplastic sheet further comprises a quantity of nano-sized particles of silver impregnated throughout said sheet;
d. wherein said quantity of nano-sized particles are comprised of a plurality of geometric crystal shapes, and wherein each respective percentage of shapes in said quantity of nano-sized particles are varied in order to minimize spatial interstices between each crystal; and,
e. wherein said covering resulting from said impregnation of silver nanoparticles and said variance in particle shape exhibits anti-microbial characteristics on both sides of said sheet and upon the exposure of its interior resulting from deformation of said sheet.

2. A covering as recited in claim 1, wherein said silver nanoparticles vary in geometric mean size of between 20 and 50 nanometers in diameter.

3. A covering as recited in claim 1, wherein said geometric crystal shapes comprise shapes of rods, prisms, spheres, and wires.

4. A covering as recited in claim 3, wherein said sheet consists of equal concentration volumes of prisms, spheres, and wires, and double the concentration volume of rods as compared to any of the other shape concentration volumes.

5. A covering as recited in claim 4, wherein said low density polyethylene comprises approximately 19 percent by weight in said sheet and said linear low density polyethylene comprises approximately 76 percent by weight in said sheet.

6. A covering as recited in claim 5, wherein said sheet includes a removable backing substrate supporting said sheet, and wherein said sheet exhibits antimicrobial properties on both sides of said sheet such that removal of said backing substrate does not substantially reduce said anti-microbial characteristics of said sheet.

7. A covering as recited in claim 1, wherein said covering includes at least two shapes of silver nanocrystals that exhibit a synergistic toxicity against a targeted pathogen.

8. A covering exhibiting anti-microbial properties for surrounding an electronic device used in a healthcare environment, comprising:
a. a flexible thermoplastic sheet configured to surround said electronic device;
b. wherein said thermoplastic sheet comprises a synthetic plastic polymer;
c. wherein said thermoplastic sheet further comprises a quantity of nano-sized particles of silver impregnated throughout said sheet;
d. wherein said quantity of nano-sized particles are comprised of a plurality of geometric crystal shapes, and wherein the respective percentage of each different shape in said quantity of nano-sized particles is varied in order to minimize spatial interstices between each crystal; and,
e. wherein said covering from said impregnation of silver nanoparticles and said variance in particle shape percentage exhibits anti-microbial characteristics on both sides of said sheet and upon the exposure of the interior of said resulting from deformation of said sheet.

9. A covering as recited in claim 8, wherein said covering exhibits a cling force between 300 grams and 500 grams, as measured in accordance with ASTM D4649.

10. A covering as recited in claim 9, wherein said particles comprise equal concentration volumes of prisms, spheres, and wires, and double the concentration volume of rods as compared to any of the other shape concentration volumes.

11. A covering as recited in claim 8, wherein said particles comprise a mixture of rod, sphere, prism, and wire shapes, and wherein said particle mixture exhibits a combined minimum inhibitory concentration level range of 0.028 PPM to 0.057 PPM by volume for said covering against a typical complement of bacterial cultures.

12. A covering as recited in claim 11, wherein said thermoplastic sheet comprises a synthetic plastic polymer selected from the group consisting of polyvinyl chloride and polyolefin .

13. A covering as recited in claim 12, wherein said sheet includes a removable backing substrate supporting said sheet, and wherein said sheet is formed into a sealable bag.

14. A covering as recited in claim 13, wherein said sheet includes at least one tab elevated from said backing for pulling and separating said sheet from said backing.

15. A covering as recited in claim 14, wherein said backing substrate is bifurcated along a center fold thereby creating a front substrate portion above said fold and a back substrate portion below said fold, and wherein said sheet on said front substrate comprises a series of perforations arranged thereon to form the shape of a cross.

16. A covering as recited in claim 15, wherein said front portion further includes a plurality of perforated concentric shapes for the selective separation of said sheet from said front substrate to conform to a size of an electronics device intended for wrapping.

17. A covering as recited in claim 8, wherein said covering includes at least two shapes of silver nanocrystals that exhibit a synergistic toxicity against a targeted pathogen.

18. A process for disrupting nosocomial infection rates in a healthcare facility, comprising the steps of:
a. providing a flexible antimicrobial thermoplastic sheet to healthcare employees configured to surround targeted electronic devices used in said healthcare facility;
i. wherein said thermoplastic sheet comprises a flexible synthetic plastic polymer film comprising a quantity of nano-sized particles of silver impregnated throughout said sheet;
ii. wherein said quantity of nano-sized particles comprises a plurality of geometric crystal shapes, and wherein the respective percentage of each different shape in said quantity of nano-sized particles is varied in order to minimize spatial interstices between each particle; and,
iii. wherein said covering from said impregnation of silver nanoparticles and said variance in particle shape percentage exhibits anti-microbial characteristics on both sides of said sheet and upon the exposure of the interior of said sheet resulting from deformation of said sheet; and,
iv. wherein said covering exhibits a cling force of at least 75 grams against itself and a targeted electronic device as measured in accordance with ASTM D5458 in order to sustain a wrapping of said cover around said targeted electronic device;
b. placing said flexible thermoplastic sheets in dispensers in readily accessible areas of said healthcare facility;
c. requiring said healthcare facility employees to utilize said thermoplastic sheets daily so that the spread of pathogens in said healthcare facility is disrupted.

19. The process as recited in claim 18, further including the step of determining through testing a minimum inhibitory concentration level for said nano-sized silver particles for a targeted group of micro-organisms and adjusting the concentration levels of said quantity of nano-sized silver particles in said antimicrobial sheet in order to meet said minimum inhibitory concentration level in said sheet.

20. The process as recited in claim 19, wherein said step of testing to determine a minimum inhibitory concentration level for said nano-sized silver particles for a targeted group of micro-organisms is tailored for a specific healthcare facility.
